(19) United States
(12) Patent Application Publication
(10) Pub. No.: US 2026/0306530 A1
(43) Pub. Date: Oct. 8, 2026

(54) NOVEL GLYCAN COMPOUNDS FOR AGE-RELATED MACULAR DEGENERATION

(71) Applicant: The Regents of the University of California, Oakland, CA (US)

(72) Inventors: Christopher B. Toomey, Poway, CA (US); Jeffrey D. Esko, San Diego, CA (US)

(21) Appl. No.: 19/476,955

(22) PCT Filed: Apr. 22, 2024
(86) PCT No.: PCT/US2024/025641

§ 371 Date: Oct. 20, 2025

Related U.S. Application Data
(60) Provisional application No. 63/497,466, filed on Apr. 21, 2023.

(51) Int. Cl. A61K 31/727; A61K 9/00; A61K 31/717; A61K 31/718; A61K 31/721; A61K 31/722; A61K 31/728; A61K 31/729; A61K 31/734; A61K 38/46; A61K 38/47; A61K 48/00; A61P 27/02; C07K 16/00

(52) U.S. Cl. CPC A61K 31/727; A61K 9/0019; A61K 9/0048; A61K 31/717; A61K 31/718; A61K 31/721; A61K 31/722; A61K 31/728; A61K 31/729; A61K 31/734; A61K 38/465; A61K 38/47; A61K 48/00; A61P 27/02; C07K 16/00; C12Y 301/06; C12Y 302/01166

(57) ABSTRACT

Compositions and methods and compositions for treating age-related macular degeneration comprising administering to a subject in need thereof an effective amount of a composition comprising an agent such as soluble glycan (e.g., heparan sulfate), to treat age-related macular degeneration by blocking the accumulation of lipoproteins within the eye, such as on Bruch's membrane.

### CROSS REFERENCE

This application claims the benefit of U.S. Provisional Application No. 63/497,466 filed Apr. 21, 2023, which application is incorporated herein by reference in its entirety.

### TECHNICAL FIELD

The present invention relates to the therapeutic treatment and/or prevention of age-related macular degeneration or it symptoms via agents such as glycans which inhibit lipoprotein binding and/or aggregation to the extracellular matrix of the eye, such as Bruch's membrane.

### BACKGROUND

Age-related macular degeneration (AMD) is a blinding eye condition that affects approximately 200 million people worldwide. There are not effective treatments for many patients.

### SUMMARY OF THE INVENTION

High impact genetic studies have suggested that Bruch's membrane (BrM) plays a key role in initiating drusenogenesis and AMD pathology. For example, heritable mutations in genes that regulate extracellular matrix (ECM) biology have been shown to cause a drusen-like phenotype that is similar to AMD, including Sorsby's macular dystrophy (TIMP3), late-onset retinal degeneration (ClqTNF5), and Doyne honeycomb retinal dystrophy (EFEMP1) [1-4]. Furthermore, in pseudoxanthoma elasticum, impaired diffusion due to progressive calcification of BrM has been shown to result in features of AMD, such as choriocapillaris flow impairment, reticular pseudodrusen, and impaired rod function [5-7]. The impaired diffusion in pseudoxanthoma elasticum is analogous to the excessive lipoprotein accumulation seen in AMD patients, which has been shown to be a diffusion barrier [8]. Additionally, the presence of multiple Complement Factor H (CFH) risk polymorphisms in protein domains that bind to HS [9-13], recent evidence that associates HTRA1 risk polymorphisms (a serine protease) with regulation of ECM metabolism, and the presence of susceptibility loci in ECM genes, including TIMP3, suggest that a primary driver of AMD pathobiology is within the ECM [14-18]. These clinical studies suggest that alterations in BrM can initiate AMD pathology.

Extensive epidemiological studies have firmly established drusen as a major contributor for the progression to advanced AMD [19, 20]. Drusen form within BrM. Substantial clinical evidence indicates that drusen are not merely an association, but instead have a pathological role in the development and progression of AMD. Mostly notably, large drusen are associated with atrophy and disruption of the outer retinal layers and diminished retinal sensitivity directly overlying the drusen [21, 22]. In addition, nascent geographic atrophy (GA) occurs overlying large drusen [23-25]. The precise spatial relationship between drusen and damage observed in the outer retinal layers strongly suggests direct involvement of drusen in AMD progression. Additionally, histopathological examination of postmortem AMD eyes has consistently revealed drusen-related changes, such as disruption and dysmorphia of the retinal pigment epithelium (RPE) immediately overlying drusen [26, 27]. It is noteworthy that drusen are characterized by high concentrations of oxidized lipoproteins and hydroxyapatite, which are believed to contribute to inflammation and serve as a focal point for the pathobiology of AMD [28-30].

Substantial evidence suggests that drusen formation results from the deposition of lipoproteins in BrM. With normal aging, lipoproteins accumulate in the inner collagenous layer (ICL) of BrM prior to the onset of drusen [31-33]. This finding highlights the early involvement of lipoprotein deposition in the pathogenesis of drusen. Secondly, basal linear deposits, which are histologically linked to AMD and drusen, have been characterized as lipoprotein deposits within the ICL of BrM [27, 34]. Thirdly, drusen form in the ICL of BrM, exhibiting a composition consisting of apolipoproteins and esterified cholesterol [32-37]. Taken together, these clinical studies suggest BrM lipoprotein accumulation and drusen formation is a driver of AMD pathobiology.

BrM is an acellular 3-layered (inner collagenous, elastic, and outer collagenous layers) ECM abutted by the RPE and choriocapillaris endothelial cell basal lamina (BL). BrM is composed of collagens (type IV collagen in RPE BL and type I and III in ICL) [38-40], laminins [39, 41], fibronectin [40] and sulfated glycosaminoglycans (GAGs), including heparan sulfate (HS) [42-45]. Given the similarities between serum and BrM lipoproteins, the sub-RPE lipoprotein retention hypothesis of drusenogenesis is analogous to the subendothelial ECM retention hypothesis in atherosclerosis [46, 47]. Studies in atherosclerosis have shown that increased retention, rather than increased influx, of lipoproteins is the primary factor driving subendothelial retention [46-50]. This retention is facilitated by the interaction of positively charged GAG binding sites on apolipoproteins and negatively charged sulfated GAGs in the arterial ECM [51-57]. The extent and type of HS regulates a wide range of biological activities including lipoprotein clearance and is altered in aging and disease [54, 58].

Now, therefore, the instant disclosure provides a groundbreaking discovery in the field of AMD, specifically focusing on the role of heparan sulfate in lipoprotein accumulation in BrM and a novel therapeutic intervention aimed at inhibiting lipoprotein accumulation, such as by using heparanoids. Additionally, the instant disclosure also provides genetic or enzymatic methods to alter BrM or RPE/choroid GAG composition, including but not limited to genetic alteration of the sulfotransferase enzymes, endogenous or exogenous heparanase or sulfatases and enzyme replacement therapy with enzymes that modify heparan sulfate in the posterior retina.

The instant invention provides in certain embodiments compositions and methods for preventing, delaying progression of, and/or treating AMD in subjects by inhibiting lipoprotein binding to and/or accumulation on an extracellular matrix of the eye (e.g., BrM). In embodiments, the compositions and methods herein are effective to prevent accumulation of lipoprotein on the extracellular matrix. In embodiments, the compositions and methods described herein are effective to remove lipoprotein from the extracellular matrix (e.g., BrM). In embodiments, the compositions and methods herein are effective to inhibit the formation of drusen on the extracellular matrix (e.g., BrM).

In embodiments, the composition effective as a therapy described herein comprises a competitive inhibitor of lipoprotein binding to glycosaminoglycans of the extracellular matrix. In embodiments, the composition comprises a soluble glycan. In embodiments, the soluble glycan is an ionic glycan (e.g., an anionic glycan).

In embodiments, the composition effective as a therapeutic described herein comprises heparan sulfate or a molecular mimetic thereof. In embodiments, the molecular mimetic of heparan sulfate is selected from alginate polymers, agarose polymers, cellulose polymers, chitosan polymers, dextran polymers, heparin polymers, hyaluronate polymers, starch polymers, carboxylated derivatives thereof, and sulfated derivatives thereof. In embodiments, the heparan sulfate or the molecular mimetic thereof has a molecular weight of at least about 5 kDa. In embodiments, the heparan sulfate or the molecular mimetic thereof has a molecular weight of at least about 20 kDa. In embodiments, the heparan sulfate or molecular mimetic thereof is a non-anticoagulant.

In embodiments, the composition effective as a therapeutic described herein comprises a heparan sulfate binding molecule. In embodiments, the heparan sulfate binding molecule is selected from surfen, protamine sulfate, a calixarene, caltrop-like multicationic molecule, and a poly-cationic peptide.

In embodiments, the composition effective as a therapeutic described herein comprises an antibody or antigen binding fragment which disrupts lipoprotein binding to the extracellular matrix (e.g., BrM).

In embodiments, the composition effective as a therapeutic described herein inhibits lipoprotein binding to the extracellular matrix by altering a structure of glycosaminoglycans (GAGs) of the extracellular matrix. In embodiments, the composition comprises a heparanase or sulfatase enzyme. In embodiments, the composition comprises a gene editing system configured to alter one or more genes encoding sulfotransferase enzymes of the subject. In embodiments, the composition comprises a gene editing system configured to alter one or more genes encoding enzymes involved in glycosaminoglycan synthesis or degradation of the subject.

In embodiments, the AMD of the subject is early, intermediate, or advanced AMD. In embodiments, the AMD of the subject comprises geographic atrophy and/or choroidal neovascularization.

In embodiments, the composition is administered to the subject intraocularly. In embodiments, the composition is administered to the vitreous body the subject. In embodiments, the composition is a pharmaceutical composition formulated for intraocular administration.

The present invention further discloses soluble glycan compositions and methods of use to remove lipoproteins trapped in the retinal or ocular extracellular matrix of a subject. The soluble glycans remove lipoprotein accumulation that occurs in the early and intermediate stages of the age-related macular degeneration. Accumulation of the glycosaminoglycan heparan sulfate is believed to cause the pathological accumulation of lipoproteins in the retinal or ocular extracellular matrix.

In embodiments, the invention provides a method of preventing or treating age-related macular degeneration comprising administering to a subject in need thereof an effective amount of a composition comprising a soluble glycan effective to remove lipoproteins from a retinal or ocular extracellular matrix of the subject.

In embodiments, the invention provides that the soluble glycan is a soluble competitive inhibitor of lipoprotein binding to extracellular matrix glycans.

In embodiments, the invention provides that the soluble glycan is a soluble heparin, or a soluble heparan sulfate. In embodiments, the invention provides that the soluble glycan is administered in a concentration ranging from 1 milligram to 100 nanograms per milliliter, or at about 10 nanograms per milliliter. In embodiments, the invention provides that the soluble glycan is administered in a concentration ranging from 10 milligram to 100 nanograms per milliliter, or at about 10 nanograms per milliliter.

In embodiments, the invention provides that the composition is administered intraocularly.

In embodiments, the invention provides that the composition is a pharmaceutical composition comprising the soluble glycan and a pharmaceutically acceptable carrier.

In embodiments, the pharmaceutical composition is used to remove pathological lipoprotein accumulation in age-related macular degeneration. In embodiments, the pharmaceutical composition is used to remove pathological lipoprotein accumulation in early and intermediate stages of age-related macular degeneration. In embodiments, the pharmaceutical composition is used to remove pathological lipoprotein accumulation in an advanced stage of age-related macular degeneration.

### BRIEF DESCRIPTION OF THE DRAWINGS

FIG. 1 shows that heparan sulfate in Bruch's membrane of human patients with Age-related Macular Degeneration (AMD). Bruch's membrane stained with H&E and immunostained for total heparan sulfate (3G10+HSase) and N-sulfated heparan sulfate (10E4). Heparan sulfate stained Bruch's membrane surrounding lipoprotein rich drusen. RPE, retinal pigmented epithelium; BrM. Bruch's membrane; CC, choriocapillaris.

FIG. 2 shows heparan sulfate composition of Bruch's membrane in human patients with Age-related Macular Degeneration (AMD). Panel A shows fundus photographs of post-mortem globes (aged control and intermediate AMD). Panel B shows age and AREDS category (AMD stage, AREDS 1, normal: AREDS 2, early AMD; AREDS 3, intermediate AMD) of post-mortem samples used for GAG analysis. Panel C shows GRIL-LC/MS analysis of total GAG (HS and CS) in macula (left) and periphery (right) of post-mortem BrM tissue from young, old and patients with early/intermediate AMD. Total HS increases in early and intermediate AMD macular BrM. Macular HS chain sulfation is shown in Panel D and disaccharide composition is shown in Panel D, which shows increases in HS N-, 2-O and 6-O sulfation in early and intermediate AMD with increases in highly sulfated disaccharides, in particular, the tri-sulfated D2S6 HS disaccharide (Panel E).

FIG. 3 shows scanning electron microscopy of human AMD BrM reveals interaction between lipoproteins and heparan sulfate. Panel A shows Ruthenium Red staining of GAGs (black staining, black arrows) in BrM. Magnified image of retained lipoprotein aggregates within drusen (Blue box). Panel B shows SEM high resolution images of lipoprotein particles in BrM associated with HS (Blue box). Panel C shows analysis of area and size of lipoprotein particles within drusen (lipoproteins, orange and Zone 1-2 demarcates HS staining in BrM). Note: the retention of lipoproteins occurs adjacent to BrM HS. RPE, retinal pigmented epithelium: BrM, Bruch's membrane; CC, choriocapillaris.

FIG. 4 shows that heparan sulfate removes lipoproteins from Bruch's membrane. The lipoprotein accumulation that occurs in Bruch's membrane with aging and age-related macular degeneration is removed by Heparin. Panel A shows Bruch's membrane from aged post-mortem donors, note there is no elution of Bruch's membrane lipoproteins with PBS control. Panel B shows incubation of aged Bruch's membrane with Heparin elutes a diverse array of lipoprotein material from Bruch's membrane. Panel C shows Western blotting of FPLC fractions show removal of ApoB100, ApoE and ApoA1 containing lipoproteins from Bruch's membrane with Heparin treatment (Bottom).

FIG. 5 shows that heparin removes lipoproteins from Bruch's membrane in ng/mL concentrations. Both ApoB100 and ApoA1 containing lipoproteins are removed from aged Bruch's membrane with heparan sulfate at concentrations ranging from 1,000 to 0.01 μg/mL. At 0.001 μg/mL the effect appears to be diminished for ApoB100 containing lipoproteins.

FIG. 6 shows human Bruch's membrane lipoprotein binding is dependent on endogenous heparan sulfate. Human Bruch's membranes were immobilized on a Quartz Crystal Microbalance (QCM) chip and treated with PBS (Panel A) or heparin lyases (Panel B) to degrade heparan sulfate. Kinetic binding experiments of HDL was performed on the human BrM QCM chip in FIGS. 6, 7 and 8. Removal of endogenous BrM heparan sulfate substantially diminishes lipoprotein binding. HDL, High Density Lipoprotein.

FIG. 7 shows human Bruch's membrane lipoprotein binding is blocked by heparin. Heparin inhibits the interaction between BrM and lipoproteins. HDL, High Density Lipoprotein.

FIG. 8 shows human Bruch's membrane lipoprotein binding is blocked by a diverse array of full-length non-anticoagulant heparan sulfates. Various heparan sulfate species (HS05, HS10, HS37, HS09 and HS30) are able to reduce and inhibit binding of lipoproteins (50 μg/mL HDL) to human BrM (Panel A). The magnitude is greatest with HS09 which is a non-anticoagulant full-length heparan sulfate species with trisulfated disaccharides (N, 2-O, 6-O sulfation) (Panel A). At higher concentrations of lipoprotein (100 μg/mL HDL) difference in inhibition between HS species (HS05 and HS09) is greater (Panel B). Injecting heparan sulfate into human BrM blocks the interaction between HDL and BrM during subsequent injections (HDL1 and HDL2 represent subsequent HDL injections for HS05 and HS09, respectively) (Panel B). HDL, High Density Lipoprotein.

FIG. 9 shows full-length non-anticoagulant heparan sulfates show increased inhibition of BrM lipoprotein binding compared to pharmaceutical grade heparin. Human Bruch's membranes were immobilized on a Quartz Crystal Microbalance (QCM) chip and kinetic binding experiments of HDL was performed in the presence of pharmaceutical grade heparin and various full-length heparan sulfate species. A wide array of full-length heparan sulfate species (HS05, HS10, HS37, HS09 and HS30) (50 g/mL) are able to inhibit binding of lipoproteins (50 μg/mL HDL) to human BrM better than equal concentrations of pharmaceutical grade heparin (50 μg/mL).

FIG. 10 shows intraocular Injection of heparan sulfate (HS) localizes to Bruch's membrane and subretinal space. PBS (left panel) or 4 μg of BODIPY-tagged HS in 1 mL PBS was intraocularly injected into the vitreous space (intravitreal, middle panel) or subretinal space (subretinal, right panel). Intravitreal BODIPY-tagged HS localizes to BrM and subretinal BODIPY-tagged HS localizes to the apical processes of the RPE. SubRet, subretinal space; RPE, retinal pigmented epithelium; BrM, Bruch's membrane; CC, choriocapillaris.

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

Heparan sulfate is a linear polysaccharide chain that is added post-translationally to proteins in almost every cell type. It comprises a 4-sugar primer sequence that is covalently linked at one end to the protein. The other end of the primer sequence is extended by the addition of repeating disaccharide units comprising N-acetyl glucosamine and glucuronic acid. Heparan sulfate chains can vary in length, from about 40 to about 300 sugar residues. The sugars that make up heparan sulfate are highly sulfated, and patterns of sulfation along the chain create negatively-charged regions that serve as binding sites for various proteins. When proteins are bound by heparan sulfate, their activity, bioavailability, and clearance may be altered. Heparin is similar to heparan sulfate; however, it is more highly sulfated and shorter in length. Non-anticoagulant forms of heparan and heparin are particularly useful in embodiments of the invention.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 50% of the recited value, or within 2% of the recited value.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U. S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which the active compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Sterile compositions are within the present disclosure, including compositions that are in accord with national and local regulations governing such compositions. Pharmaceutical compositions including the active ingredient, alone or in combination with other active ingredient(s), described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. An active compound, alone or in combination with other active ingredient(s), described herein, and preferably in the form of a pharmaceutical composition, may be administered by a suitable route of delivery, such as oral, parenteral, rectal, nasal, topical, or ocular routes, or by inhalation. In some embodiments, the compositions are formulated for intravenous or oral administration.

For parenteral use, including intraocular, intravenous, intramuscular, intraperitoneal, intranasal, or subcutaneous routes, a glycan, alone or in combination with other active ingredient(s), may be provided in sterile aqueous solutions or suspensions, buffered to an appropriate pH and isotonicity or in parenterally acceptable oil. Suitable aqueous vehicles can include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 g/kg/minute of agent admixed with a pharmaceutical carrier over a period ranging from several minutes to several days.

One of ordinary skill in the art may modify the formulations within the teachings of the specification to provide numerous formulations for a particular route of administration. In particular, a glycan, alone or in combination with other active ingredient(s), may be modified to render them more soluble in water or other vehicle. It is also well within the ordinary skill of the art to modify the route of administration and dosage regimen of the compositions, alone or in combination with other active ingredient(s), in order to manage the pharmacokinetics of the present compositions for maximum beneficial effect in a patient.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

### Treatments of AMD

In embodiments provided herein are methods for preventing, delaying the progression of, or treating age-related macular degeneration (AMD) using compositions described herein. The compositions described herein are those which are capable of reducing lipoprotein binding to an extracellular matrix (ECM) of the eye, preferably to Bruch's membrane (BrM). The treatments of the instant disclosure can be successful in the prevention of AMD, the prevention of progression of AMD, delaying the progression of AMD, the treatment of AMD, the reversal of AMD, and/or the alleviation or reduction of one or more symptoms of AMD.

In embodiments, the methods of treating AMD herein are based around the newly observed discovery that interrupting the binding of lipoproteins to gly cosaminoglycans (GAGs) of the ECM (e.g., BrM) can reduce lipoprotein retention to the ECM. This interruption can thereby reduce, eliminate, or even reverse the formation of drusen in the eye which can drive the development of AMD in patients.

In embodiments, the compositions described herein can be useful in the treatment of a variety of stages of AMD. In embodiments, the compositions and methods described herein are useful in the treatment of AMD are used to treat early, intermediate, or advanced AMD. In embodiments, the AMD in a subject undergoing treatment as described herein has early or intermediate AMD. In embodiments, the subject has advanced AMD. In embodiments, the advanced AMD comprises one or both of geographic atrophy and/or choroidal neovascularization.

In embodiments, the compositions and/or methods described herein are effective to prevent accumulation of lipoprotein on the ECM (e.g., the BrM). In embodiments, accumulation of lipoprotein on the ECM by at least 10%, 20%, 30%, 40%, 50%, 60%, 70%, 80%, or 90% relative to a subject in which no intervention is delivered. In embodiments, the compositions and/or methods described herein are effective to remove lipoproteins (including aggregates thereof) from the ECM, such that at least 10%, 20%, 30%, 40%, 50%, 60%, 70%, 80%, or 90% of the lipoproteins are removed. In embodiments, the ECM is preferably the BrM.

In embodiments, the compositions and/or methods described herein are effective to inhibit the formation of drusen on the Bruch's membrane of the subject. In embodiments, formation of drusen in the subject is reduced by at least 10%, 20%, 30%, 40%, 50%, 60%, 70%, 80%, or 90% (e.g., as compared to a comparable subject without any intervention). In embodiments, the compositions and/or methods described herein are effective to reduce the number of drusen formed or the total volume of drusen formed in the subject (e.g., the composition is effective to reduce/reverse the number of drusen or the volume of drusen in the subject). In embodiments, the composition is effective to reduce the number of drusen by at least 10%, 20%, 30%, 40%, 50%, 60%, 70%, 80%, or 90%. In embodiments, the composition is effective to reduce total drusen volume by at least 10%, 20%, 30%, 40%, 50%, 60%, 70%, 80%, or 90%.

In embodiments, the compositions and/or methods described herein are effective to improve or slow the progression of AMD as determined based on Age-Related Eve Disease Study (AREDS) categories (see. e.g., [Age-Related Eye Disease Study Research G. The Age-Related Eye Disease Study (AREDS): design implications. AREDS report no. 1. Control Clin Trials. 1999; 20(6):573-600) (AREDS)]). AREDS categories include category 1 (defined as a few (1-15), small (<63 μm), or no drusen and without pigment changes); category 2 (early AMD characterized by several small, few intermediate-sized (63-124 μm) drusen, and/or pigmentary changes in one or both eyes); category 3 (intermediate AMD characterized by extensive (20 soft or 65 hard without any soft) intermediate-size drusen, one large (>125 μm) drusen, and/or geographic atrophy not involving the macula in one or both eyes); and category 4 (advanced unilateral AMD consisting of the advanced dry form with geography atrophy involving macula or the exudative form with choroidal neovascularization in one eye).

In embodiments, the compositions described herein are administered directly to the relevant tissue of the subject (i.e., to the eye of the subject). In embodiments, the composition described herein (including pharmaceutical compositions described herein) is administered intraocularly. In embodiments, the intraocular administration is intravitreal administration.

In embodiments, the desired therapeutic effect is achieved following a plurality of doses (e.g., 2, 3, 4, 5, 6, 7, 8, 9, 10, or more doses) of the composition. In embodiments, the plurality of doses are administered according to a defined dosing regimen (e.g., once daily, three times per week, twice per week, once per week, every two weeks, every three weeks, every four weeks, monthly, etc.). Accordingly, the administration of multiple doses of therapeutically effective amounts of a composition described herein (e.g., one comprising a glycan such as heparan sulfate or another compound described herein) is expressly considered.

### Compositions Effective for Inhibition of Lipoprotein Binding to ECM

The compositions useful in methods of treatment as described herein contain active agents which are effective to inhibit lipoprotein binding to glycosaminoglycans present on eye ECMs, in particular BrM. The active agent can be selected from a wide variety of potential agents, including those described in more detail below. In embodiments, the composition comprises a competitive inhibitor of lipoprotein binding to glycosaminoglycans of the extracellular matrix (e.g., BrM).

### Glycans

In embodiments, the composition can comprises one or more glycan molecules as the active agent which is effective in inhibiting lipoprotein binding to ECMs (e.g., BrM). In embodiments, the glycan is a soluble glycan. In embodiments, the glycan is an ionic glycan. In embodiments, the glycan is cationic, anionic, or zwitterionic. In embodiments, the glycan is an anionic glycan. Non-limiting examples of glycans of the instant disclosure include alginate polymers, agarose polymers, cellulose polymers, chitosan polymers, dextran polymers, heparan polymers, hyaluronate polymers, and starch polymers. In embodiments, the glycan is a derivatized glycan, such as a carboxylated glycan (e.g., carboxymethylated) or a sulfated glycan (e.g., N-sulfated aminoglycans). In embodiments, the glycan is a biodegradable glycan. In embodiments, the glycan is a non-anticoagulant (e.g., does not possess significant anticoagulant activity).

In embodiments, the glycan is of a desired size for proper functioning in vivo (e.g., the glycan is of a minimum molecular weight in order to demonstrate its therapeutic effect). In embodiments, the glycan has a molecular weight of at least 3.5 kDa, 5 kDa, 7.5 kDa, 10 kDa, 12.5 kDa, 15 kDa, 17.5 kDa, or 20 kDa. In embodiments, the glycan has a molecular weight of at least 5 kDa. In embodiments, the glycan has a molecular weight of at least 20 kDa In embodiments, the glycan has a molecular weight from about 3.5 kDa to about 100 kDa, about 3.5 kDa to about 75 kDa, from about 3.5 kDa to about 50 kDa, from about 3.5 kDa to about 40 kDa, from about 5 kDa to about 100 kDa, about 5 kDa to about 75 kDa, from about 5 kDa to about 50 kDa, from about 5 kDa to about 40 kDa, from about 10 kDa to about 100 kDa, about 10 kDa to about 75 kDa, from about 10 kDa to about 50 kDa, from about 10 kDa to about 40 kDa, from about 15 kDa to about 100 kDa, about 15 kDa to about 75 kDa, from about 15 kDa to about 50 kDa, from about 15 kDa to about 40 kDa, from about 20 kDa to about 100 kDa, about 20 kDa to about 75 kDa, from about 20 kDa to about 50 kDa, or from about 20 kDa to about 40 kDa. In embodiments, the glycan has a molecular weight of at most about 100 kDa, 75 kDa, 50 kDa, or 40 kDa.

In embodiments, the composition comprises heparan sulfate or a molecular mimetic of heparan sulfate as the active agent (e.g., a glycan having a similar ability to bind to substrates of heparan sulfate). In embodiments, the molecular mimetic of heparan sulfate is selected from alginate polymers, agarose polymers, cellulose polymers, chitosan polymers, dextran polymers, heparan polymers, hyaluronate polymers, starch polymers, carboxylated derivatives thereof, and sulfated derivatives thereof. In embodiments, the heparan sulfate mimetic is a sulfated derivative of an alginate polymer, agarose polymer, cellulose polymer, chitosan polymer, dextran polymer, heparan polymer, hyaluronate polymer, or starch polymer. In embodiments, the molecular mimetic of heparan sulfate is a non-anticoagulant. In embodiments, the molecular mimetic is dextran sulfate or alginate. In embodiments, the molecular mimetic is dextran sulfate. In embodiments, the molecular mimetic is alginate.

In embodiments, the heparan sulfate or molecular mimetic of heparan sulfate has a molecular weight of at least 3.5 kDa, 5 kDa, 7.5 kDa, 10 kDa, 12.5 kDa, 15 kDa, 17.5 kDa, or 20 kDa. In embodiments, the heparan sulfate or molecular mimetic of heparan sulfate has a molecular weight of at least 5 kDa. In embodiments, the heparan sulfate or molecular mimetic of heparan sulfate has a molecular weight of at least 20 kDa In embodiments, the heparan sulfate or molecular mimetic of heparan sulfate has a molecular weight from about 3.5 kDa to about 100 kDa, about 3.5 kDa to about 75 kDa, from about 3.5 kDa to about 50 kDa, from about 3.5 kDa to about 40 kDa, from about 5 kDa to about 100 kDa, about 5 kDa to about 75 kDa, from about 5 kDa to about 50 kDa, from about 5 kDa to about 40 kDa, from about 10 kDa to about 100 kDa, about 10 kDa to about 75 kDa, from about 10 kDa to about 50 kDa, from about 10 kDa to about 40 kDa, from about 15 kDa to about 100 kDa, about 15 kDa to about 75 kDa, from about 15 kDa to about 50 kDa, from about 15 kDa to about 40 kDa, from about 20 kDa to about 100 kDa, about 20 kDa to about 75 kDa, from about 20 kDa to about 50 kDa, or from about 20 kDa to about 40 kDa. In embodiments, the heparan sulfate or molecular mimetic of heparan sulfate has a molecular weight of at most about 100 kDa, 75 kDa, 50 kDa, or 40 kDa.

In embodiments, the composition comprises heparan sulfate as the active agent. The heparan sulfate can have a wide variety of ranges of sulfation at various positions. For example, the heparan sulfate can comprise a degree of N-sulfation (defined as the number sulfated nitrogens/100 disaccharides) of anywhere from 0 to 100 (e.g., 0-10, 10-20, 20-30, 30-40, 40-50, 50-60, 60-70, 70-80, 80-90, 90-100, etc.). In embodiments, the degree of N-sulfation is from about 80-100. In embodiments, the degree of N-sulfation is about 0-10. In embodiments, the heparan sulfate comprises a degree of O-sulfation of from about 1 to about 200 (e.g., 0-10, 100-200, 50-150, etc.). In embodiments, the heparan sulfate comprises a degree of 2-O-sulfation of anywhere from 0 to 100 (e.g., about 0, 10, 20, 30, 40, 50, 60, 70, 80, 90, or 100). In embodiments, the heparan sulfate a degree of 6-O-sulfation of anywhere from 0 to 100 (e.g., about 0, 10, 20, 30, 40, 50, 60, 70, 80, 90, or 100). In embodiments, the heparan sulfate comprises a degree of 3-O-sulfation of 0 to 10 (e.g., 1, 2, 3, 4, 5, 6, 7, 8, 9, or 10). Preferably, the overall degree of sulfation of the heparan sulfate is at least 1, 2, or 3.

In embodiments, the heparan sulfate has a molecular weight of at least 3.5 kDa, 5 kDa, 7.5 kDa, 10 kDa, 12.5 kDa, 15 kDa, 17.5 kDa, or 20 kDa. In embodiments, the heparan sulfate has a molecular weight of at least 5 kDa. In embodiments, the heparan sulfate has a molecular weight of at least 20 kDa. In embodiments, the heparan sulfate has a molecular weight from about 3.5 kDa to about 100 kDa, about 3.5 kDa to about 75 kDa, from about 3.5 kDa to about 50 kDa, from about 3.5 kDa to about 40 kDa, from about 5 kDa to about 100 kDa, about 5 kDa to about 75 kDa, from about 5 kDa to about 50 kDa, from about 5 kDa to about 40 kDa, from about 10 kDa to about 100 kDa, about 10 kDa to about 75 kDa, from about 10 kDa to about 50 kDa, from about 10 kDa to about 40 kDa, from about 15 kDa to about 100 kDa, about 15 kDa to about 75 kDa, from about 15 kDa to about 50 kDa, from about 15 kDa to about 40 kDa, from about 20 kDa to about 100 kDa, about 20 kDa to about 75 kDa, from about 20 kDa to about 50 kDa, or from about 20 kDa to about 40 kDa. In embodiments, the heparan sulfate has a molecular weight of at most about 100 kDa, 75 kDa, 50 kDa. or 40 kDa.

The heparan sulfate can be derived from a variety of sources. In embodiments, the heparan sulfate is derived from a cell line selected or engineered to generate the heparan sulfate with desired properties (e.g., degree of sulfation, size, composition, etc.). Preferably, the cell line generates heparan sulfate which is a non-anticoagulant, as anticoagulation can in certain instances be incompatible with intraocular delivery due to bleeding within the eye following delivery. The cell lines can be of a variety of types, but are preferably mammalian cell lines. The cell lines can also be engineered to alter genes implicated in the assembly or modification of heparan sulfate in order to tailor therapeutic heparan sulfate to have desired properties. Engineering of such cells can comprise modification in genes implicated in the generation of heparan sulfate within the cells. Exemplary genes implicated in the biosynthesis of heparan sulfate and modification thereof are known in the art and described in, for example, Annaval et al., “Heparan Sulfate Proteoglycans Biosynthesis and Post Synthesis Mechanisms Combine Few Enzymes and Few Core Proteins to Generate Extensive Structural and Functional Diversity,” Molecules. 2020 September: 25(18): 4215.

In embodiments, the glycan (e.g., heparan sulfate) is administered in an amount effective to exhibit the desired activity. In embodiments, an amount of glycan (e.g., heparan sulfate) sufficient to achieve an intraocular concentration of at least 0.1 microgram/mL, 0.2 microgram/mL, 0.5 microgram/mL, 1 microgram/mL, 5 microgram/mL, 10 microgram/mL, 25 microgram/mL, 50 microgram/mL, 75 microgram/mL, 100 microgram/mL, 250 microgram/mL, 500 microgram/mL, or 1000 microgram/mL is administered (e.g., administered as a single dose).

### Heparin and Heparan Sulfate Binders

In embodiments, alternative to the glycans discussed above, an active agent of a composition which is effective in inhibiting lipoprotein binding to ECMs (e.g., BrM) can comprise a molecule which binds to heparin or heparan sulfate. Preferably, such compounds used as active agents are capable of blocking interactions between heparin and/or heparan sulfate with lipoproteins such as high density lipoprotein (HDL). Without being bound by any theory, it is predicted that such molecules could be similarly effective to the exemplified therapeutic compositions provided herein in inhibiting lipoprotein binding to ECMs by themselves binding to the ECMs and blocking the binding sites of the ECMs bound by the lipoproteins. Thus, lipoprotein binding to ECMs (and, optionally and desirably, subsequent aggregation of the lipoproteins) is reduced or, in some instances, even reversed, thus treating, preventing, or slowing/halting the progression of AMD in the patient.

Non-limiting examples of such heparin and/or heparan sulfate binding compounds include surfen, protamine sulfate, a calixarene, caltrop-like multicationic molecule, poly-cationic peptides, and derivatives and analogs thereof. In embodiments, the active agent is a heparin or heparan sulfate antagonist. In embodiments, the composition comprises surfen or a surfen analog as the active agent. Surfen and its analogs are well known in the art and examples can found in, for example, Weiss et al., “Small molecule antagonists of cell-surface heparan sulfate and heparin-protein interactions,” Chem. Sci., 2015, 6, 5984-5993. In embodiments, the composition comprises protamine sulfate as the active agent. In embodiments, the composition comprises a calixarene as the active agent. In embodiments, the composition comprises a caltrop-like multicationic molecule, such as those described in, for example, Zong et al., “Caltrop-like Small-Molecule Antidotes That Neutralize Unfractionated Heparin and Low-Molecular-Weight Heparin In Vivo,” J. Med. Chem. 2024, 67, 5, 3860-3873. In embodiments, the composition comprises a poly-cationic peptide as the active agent.

### Antibodies

In embodiments, the active agent of a composition as described herein comprises an antibody or an antigen binding fragment thereof which is effective in inhibiting lipoprotein binding to ECMs (e.g., BrM). In embodiments, the composition comprises an antibody or antigen binding fragment which disrupts lipoprotein binding to the extracellular matrix. In embodiments, the antibody or antigen binding fragment thereof is capable of binding to one or more of heparin, heparan sulfate, or lipoproteins. Preferably, the antibody or antigen binding fragment binds to one of these groups at or near a site which is implicated in the binding of lipoproteins (e.g., HDL, LDL, etc.) to heparin and/or heparan sulfate (or other glycosaminoglycans of the BrM) in order to most efficaciously disrupt the interaction and thus treat the AMD. In embodiments, the antibody or antigen binding fragment thereof is specific for heparin. In embodiments, the antibody or antigen binding fragment thereof is specific for heparan sulfate. In embodiments, the antibody or antigen binding fragment thereof is specific for a lipoprotein, such as an apoliporptoein (e.g., chylomicrons, chylomicron remnants, VLDL, VLDL remnants (IDL), LDL, HDL, and/or Lipoprotein A).

The antibody or antigen binding fragment can be of any format known in the art. Representative antigen binding fragments include, but are not limited to, a Fab, a Fab′, a F(ab′)2, a bispecific F(ab′)2, a trispecific F(ab′)2, a variable fragment (Fv), a single chain variable fragment (scFv), a dsFv, a bispecific scFv, a variable heavy domain, a variable light domain, a variable NAR domain, bispecific scFv, an AVIMERR, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination ofVL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides), and the like. In some preferred embodiments, an antigen binding fragment will comprise both a variable heavy domain and a heavy light domain.

### Glycosaminoglycan (GAG) Modification

Under an alternative approach (or one used in conjunction with the active agents discussed supra), a composition described herein effective in inhibiting lipoprotein binding to ECMs (e.g., BrM) works by altering the structure of ECMs within or near the BrM (e.g., ECMs of BrM or RPE/choroid). By altering the GAG structure within the eye (e.g., in or near the posterior retina), buildup of lipoproteins (including aggregation thereof) and subsequent drusen formation can be minimized, reduced, or eliminated. Under such an approach, an active agent capable of altering the GAG structure of BrM or RPE/choroid could be delivered to the eye. Non-limiting examples of how to accomplish such an approach include the bolus delivery of a heparanase or sulfatase enzyme (either the same as those of the subject or an engineered version thereof) and/or by editing the genes of local tissue of the subject to express such a heparanase or sulfatase enzyme (e.g., by delivering a gene editing system to the relevant tissue of the subject). In embodiments, the composition comprises a heparanase or sulfatase enzyme. In embodiments, the composition comprises a gene editing system configured to alter one or more genes encoding sulfotransferase enzymes of the subject.

### Pharmaceutical Compositions

In further embodiments described herein are pharmaceutical compositions comprising an active agent provided herein effective to inhibit lipoprotein binding to an extracellular matrix of the eye (e.g., a soluble glycan such as heparan sulfate), and a pharmaceutically acceptable carrier. In preferred embodiments, the pharmaceutically acceptable carrier is ophthalmically acceptable (e.g., causing minimal irritation and/or damage to the eye upon delivery). In preferred embodiments, the pharmaceutical composition is formulated for intraocular administration (e.g., intravitreal administration).

A more complete understanding can be obtained by reference to the following specific examples which are provided herein for purposes of illustration only, and are not intended to limit the scope of the invention.

### EXAMPLES

### Methods

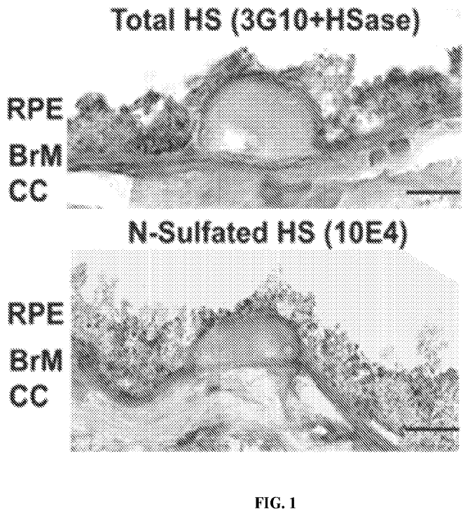

Heparan Sulfate Immunohistochemistry: Post-mortem human tissue isolated from a 90-year-old male with early age-related macular degeneration from the San Diego Eye Bank with a post-mortem interval of 16 hours. The posterior pole was isolated and a 4 mm punch of Retina/RPE/Choroid was obtained from the macula and embedded in OCT and flash frozen in a small stainless-steel bowl with a 2-methyl-butane and dry ice pellet slurry and then transferred to −80 degrees for storage. 10-micron cross-sections were cut with assistance from the La Jolla Institute for Immunology Histology Core. Frozen sections where air dried, fixed with 95% ethanol and treated with PBST (PBS with 0.1% Tween) to remove endogenous peroxidase and then blocked with 3% BSA. Heparin lyase digestion was performed with Hep I/II/III 5 mM/mL for 1 hour at room temperature as a control. The tissue was then treated with primary antibody 10E4 or 3G10 to stain for N-sulfated HS chains or HS stubs [59]. Slides were washed and then treated with secondary donkey anti-mouse IgM peroxidase and anti-mouse IgG peroxidase, respectively, at 1:2000. DAB and hematoxylin treatment were performed and then slides were imaged on a Zeiss AxioLab Microscope at 100× magnification (FIG. 1).

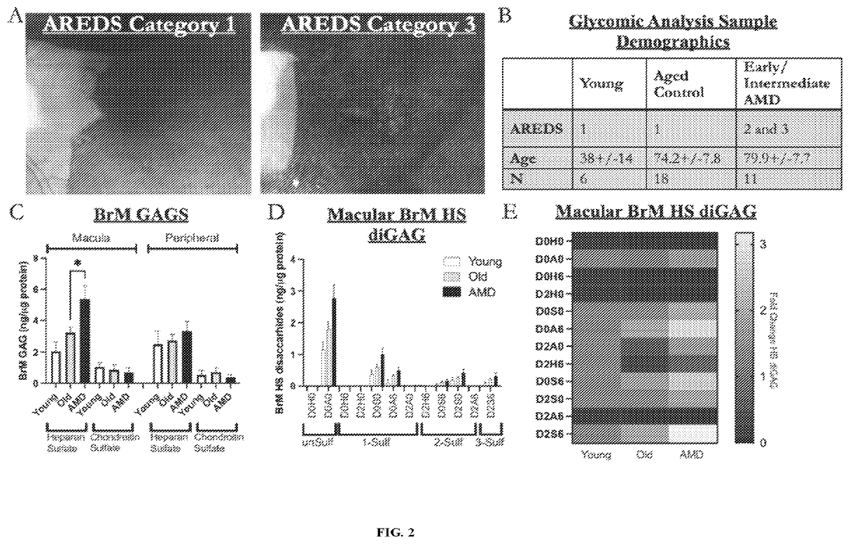

Glycan Reductive Isotope Labeling and Liquid Chromatography/Mass spectrometry of Glycosaminoglycans (GAG): Donor eves were procured from the San Diego Eye Bank from ages 25-45 years old and 65-90 years old, including patients with AMD. A short (<12-hour) death-enucleation interval and receiving interval (<24 hours) was used to ensure GAG stability. After dissection, high-resolution, color and digital fundus photographs were taken of the posterior pole (FIG. 2, Panel A). AMD grading was performed according to the 9-step Minnesota Grading scale and AREDS categories (FIG. 2, Panels A and B) [60, 61]. Macular and peripheral punches (6 mm diameter) of the RPE/BrM/choroid complex were performed. BrM was isolated from the RPE/BrM/choroid complex by microscopic dissection [62]. Clinical data including regarding smoking status and past ocular history was documented. PCR genotyping was performed for the common CFH (rs1061170) and HTRA1/ARMS2 (rs11200638). GAGs were quantified by GRIL-LC/MS [63]. Briefly, GAGs were isolated with DEAE Sepharose column, and the reducing end of lyase-generated disaccharides were tagged with aniline-containing stable ¹²C₆ isotope. Samples will be mixed with ¹³C₆ aniline tagged GAG disaccharides standards and quantified with LC/MS (FIG. 2. Panels B-E). Individual disaccharides were quantified relative to known amounts of mass-tagged standards, summed to give the total weight of GAG and disaccharide nanograms per microgram of protein (as shown in FIG. 2, Panels C and D). It was calculated that N=5 per group was needed to detect an anticipated 2.5 standard deviation change in BrM HS content based on our preliminary data (FIG. 2, Panels C-E). Statistical testing was performed using ANOVA and GraphPad software.

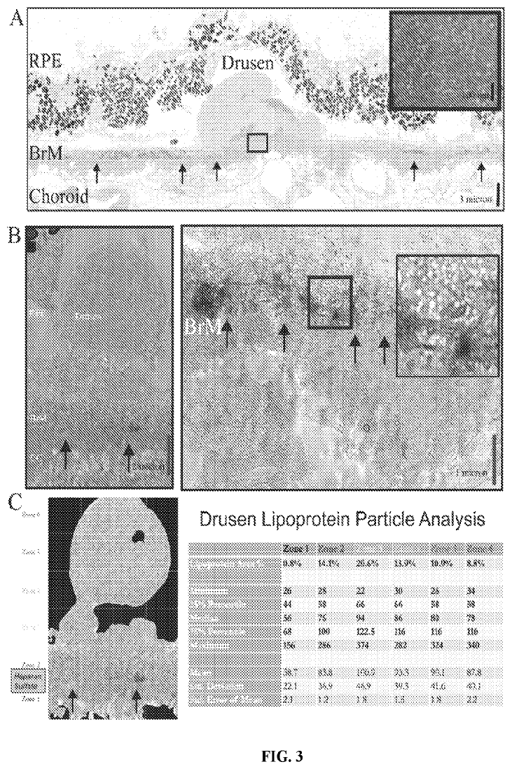

Scanning Electron Microscopy Analysis of GAGs and Lipoproteins in Human BrM: Post-mortem human tissue isolated from an 83 year old female and a 91 year old male with early age-related macular degeneration from the San Diego Eye Bank with a post-mortem interval of less than 5 hours. The globe was immediately preserved in 4% paraformaldehyde. The posterior pole was isolated, and a 2 mm punch of Retina/RPE/Choroid was obtained from the macula and fixed in 2.5% glutaraldehyde, 4% paraformaldehyde in 100 mM sodium cacodylate buffer. Samples were rinsed and stored in a cryoprotectant 4:3:3; 1×PBS:glycerol:ethylene glycol. A foveal sample was bisected and embed in agarose, and 50-200 μm vibratome sections were cut. The most preserved sections were then used for staining and fixation with dehydrate in ascending concentrations of ethanol and embed in epoxy resin. The samples were then post-fixed with 1.2% glutaraldehyde with 0.05% ruthenium red and 0.06M sodium cacodylate buffer, then further post-fixation with 1.6% osmium with 0.05% ruthenium red 0.06M sodium cacodylate buffer. 60-80 nm ultrathin sections were then cut using a Leica UC7/FC7 ultramicrotome (Waitt Advanced Biophotonics Core, Salk Institute). SEM imaging was performed on the Carl Zeiss SIGMA Variable Pressure Field Emission Gun Scanning Electron Microscope equipped with an ATLAS montage imaging module and the Shuttle and Find correlative microscopy navigation module to generate images and high-throughput workflow (Waitt Advanced Biophotonics Core, Salk Institute). SEM sections with drusen deposits in BrM were analyzed using manual segmentation with ImageJ to determine the distribution of lipoprotein particle diameter and percent area of lipoprotein particles in various zones. Lipoprotein particles were identified based on the characteristic electron lucent core morphology. Ruthenium red staining was used to identify GAGs on SEM images. 1 micron² zones were created for analysis of GAG lipoprotein spatial relationship where the border between Zone 1 and Zone 2 is defined as the 1 micron² posterior and anterior to the GAG staining in BrM (FIG. 3, Panel C). Each addition zone is 1 microns² anterior (FIG. 3, Panel C). Statistical analysis of lipoprotein particle size and spatial relationship between GAGs was performed using was performed using ANOVA and GraphPad software.

Removal of BrM Lipoproteins with Heparin: Post-mortem human tissue isolated from aged donors>75 years of age from the San Diego Eye Bank with a post-mortem interval<24 hours. Macular and peripheral punches (6 mm diameter) of the RPE/BrM/choroid complex were performed. BrM was isolated from the RPE/BrM/choroid complex by microscopic dissection [62]. The BrM were washed with PBS 3 times and careful dissection was performed to remove all visible choroidal vessels and blood contamination. BrM were minced with a single edge razor blade (Electron Microscopy Sciences) and then treated overnight at 4 degrees with either PBS or PBS with heparan sulfate (1 microgram/mL) on low speed orbital shaker (Corning). The samples were concentrated to 250 microliters and injected into a BIO RAD NGC Chromatography FPLC system (UCSD FPLC Core). Cholesterol, Cholesterol Esterase and Triglyceride levels for each FPLC fractions were performed using the Amplex Red Cholesterol Assay (Invitrogen) and High Sensitivity Triglyceride kits (Sigma-Aldrich). Fractions were pooled and concentrated for western blot analysis of ApoA1 and ApoB1 to determine their apolipoprotein content using lab standard techniques. Briefly, every 3 fractions were pooled, concentrated, loaded on to a NuPAGE Bis-Tris gel and transferred to PVDF membrane. The western blot was incubated with primary antibodies for Rabbit anti-ApoA1 and biotinylated anti-ApoB and secondary antibodies Goat anti-Rabbit-800 and streptavidin-680 then images with the Li-Cor Odyssey DLx imaging system for quantification of proteins.

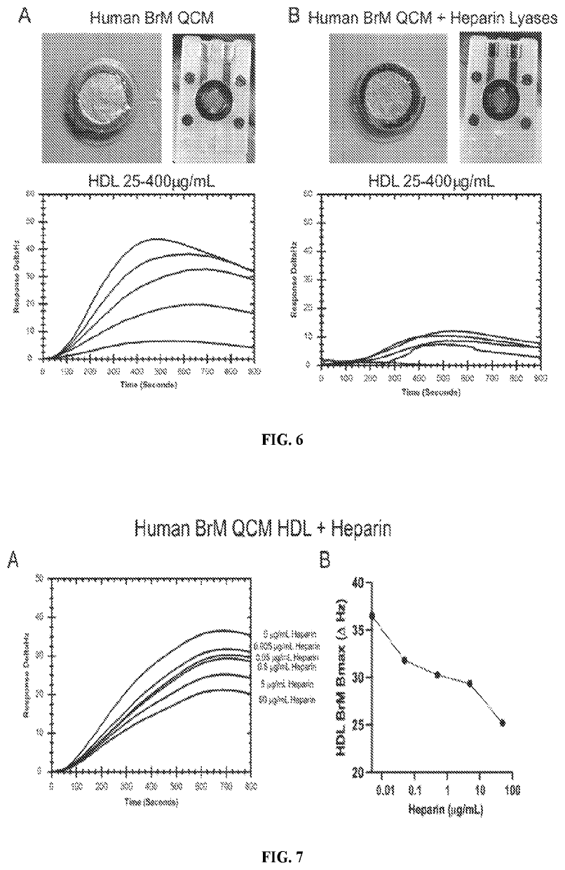

Quartz Crystal Microbalance of BrM using Heparin and Full-length HS species: BrM was isolated using gentle mechanical dissection of the overlying RPE and underlying choroid using Finesse Maxgrip forceps and a Finesse Flexloop (Alcon, California) and gentle decellularization is performed with hypotonic solutions. 5 mm tissue biopsy punch (Miltex, New Jersey) of isolated BrM were positioned on gold coated Attana QCM biosensors (Attana Life Science and Diagnostics, Sweden). Overnight treatment with a mixture of heparin lyases I/II/III BrM punches was used to control for heparan sulfate specific interactions. The QCM biosensors were dehydrated overnight to immobilize the BrM ECM (FIG. 6, Panels A and B (top)). The QCM-BrM biosensor and QCM-BrM Biosensors treated with heparin lyases were analyzed in parallel on the Attana QCM instrument to examine the kinetics of lipoprotein binding to BrM. After overnight stabilization, lipoprotein analytes in PBS were injected at 10 μL/s (low flow rate). The delta Hz was measured for 30 minutes generating long association and disassociation phases (FIG. 6). Kinetic binding curves were used to calculate the apparent affinity (K_{D}) and association (kₐ) and disassociation (k_{d}) rates for various lipoprotein species and lipoprotein mixtures (FIGS. 6-9).

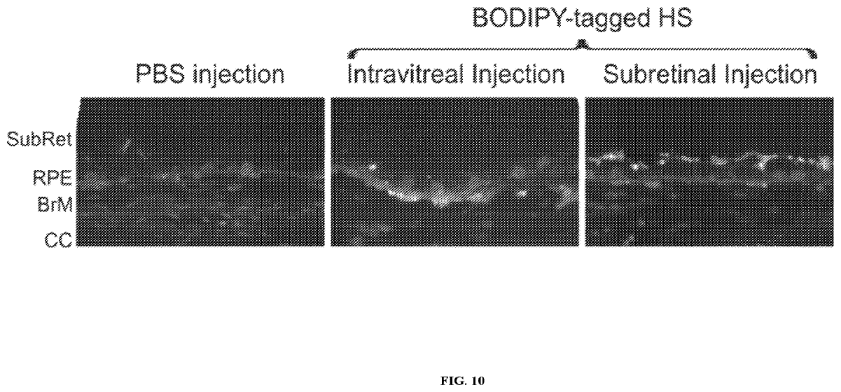

In vivo pharmacokinetics of BODIPY-tagged heparin: Pharmaceutical grade heparin was aminated with 400 mM diaminopropane in sodium acetate buffer, then reduced with sodium cyanoborohydride and then purified using DEAE column and desalted with PD10 column. The aminated heparin was reacted with succinimidyl ester BODIPY™ FL-X dye (ThermoFisher) and separated by size exclusion chromatography (PD10). BODIPY-labeled heparin was quantified using a carbazole assay and injected at 4.4 g/mL (1 μL) into the intravitreal and subretinal space in 15-20-week-old mice wild type. Twenty-four hours later mice were sacrificed, and eyes were enucleated. A small anterior chamber incision was performed, and the globe was fixed with 4% paraformaldehyde for 24 hours and then transferred to isopropanol. Fixed tissue was embedded in paraffin and cross-sections were cut with assistance from the La Jolla Institute for Immunology Histology Core. Paraffin sections were deparaffinized with xylene and ethanol. Slides were washed and nuclei were stained with Hoechst dye and mounted. Slides were imaged on a Zeiss Observer Z1 inverted fluorescent microscope at 40× magnification (FIG. 10).

### Results

Heparan Sulfate is increased in AMD BrM: Given the potential role of HS in trapping lipoproteins in AMD BrM, it was sought sought to localize and quantify the content of HS in BrM in AMD. HS is present in AMD BrM and encapsulates drusen in AMD patients based on immunohistochemistry using 3G10 mAb to detect HS stub epitopes generated by heparin lyase digestion (FIG. 1, top). Interestingly, using 10E4 antibody that specifically recognizes N-sulfated HS epitope there does not appear to be significant N-sulfation underlying drusen (FIG. 1, bottom). It is also noteworthy that no HS staining was present within the core of the drusen (FIG. 1).

Glycosaminoglycans were extracted human post-mortem BrM tissue and quantified by GRIL LC/MS analysis of disaccharides liberated by heparin lyase digestion. Fundus photographs were taken of all post-mortem globes to stage AMD and representative fundus photos from an aged control (right) and intermediate AMD (left) are shown in FIG. 2, Panel A. The table present in FIG. 2, Panel B shows the composition and age-range for the post-mortem globes used in the GRIL LC/MS analysis. GRIL-LC/MS analysis of total GAG (HS and CS) in macula (left) and periphery (right) of post-mortem BrM tissue from young, old and patients with early/intermediate AMD shows that the total HS increases in early and intermediate AMD macular BrM (FIG. 2, Panel C). Macular HS chain sulfation (FIG. 2. Panel D) and disaccharide composition (FIG. 2, Panel E), shows increases in HS N-, 2-0 and 6-0 sulfation in early and intermediate AMD with increases in highly sulfated disaccharides, particularly the tri-sulfated D2S6 HS disaccharide.

Heparan Sulfate interacts with lipoprotein particles in AMD BrM: Scanning electron microscopy with Ruthenium Red staining was performed to analyze the interaction between HS and lipoprotein particles in AMD BrM. Ruthenium Red staining of GAGs (black staining, black arrows) in BrM is shown in FIG. 3, Panel A. A magnified image of retained lipoprotein aggregates within drusen is shown (Blue box). Representative SEM sections are shown to highlight the interaction between lipoprotein particles and the Ruthenium Red staining (black) (FIG. 3, Panels A-B). To determine the spatial relationship between lipoprotein particles and HS, manually segmented lipoprotein particles were prepared and analyzed their spatial relationship relative to BrM HS (FIG. 3, Panel C). Analysis of the area and size of lipoprotein particles within drusen (lipoproteins, orange and Zone 1-2 demarcates HS staining in BrM) shows that the retention of lipoproteins occurs adjacent to BrM HS (FIG. 3, Panel C).

Heparin and Full-Length Heparan Sulfate Species Inhibit Lipoprotein Binding in BrM: In addition to describing the increase in HS that occurs in AMD BrM (FIG. 1-2) and the spatial relationship between HS and lipoproteins using scanning electron microscopy (FIG. 3), a quartz crystal microbalance biosensor (QCM) analysis was used to assess whether the interaction between BrM and lipoproteins depends on heparan sulfate (FIG. 6). QCM biosensor allows for quantitative analysis of binding kinetics between complex structures. Here BrM from post-mortem human donors was immobilized to gold plated QCM biosensor chips and injected purified plasma HDL (100 μg/mL) at 10 ml/sec (FIG. 6, top). To determine the role of heparan sulfate on lipoprotein binding to BrM some samples were treated with heparin lyase, which removed the majority of binding (FIG. 6).

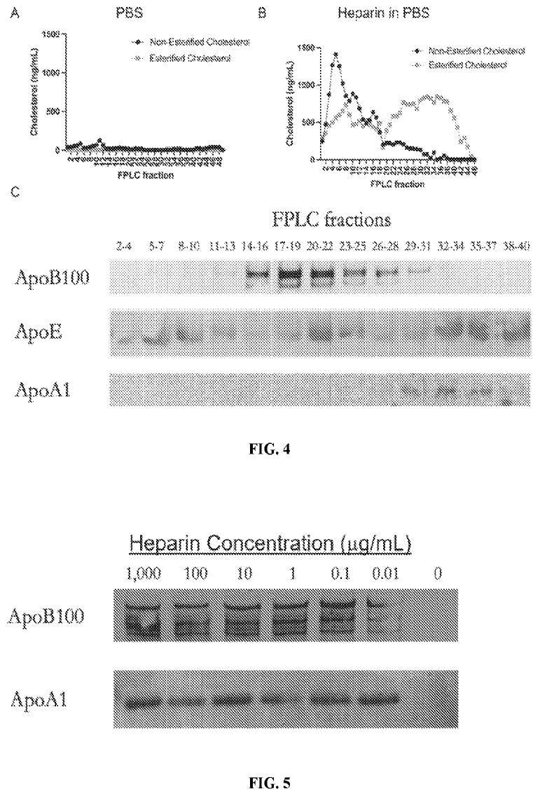

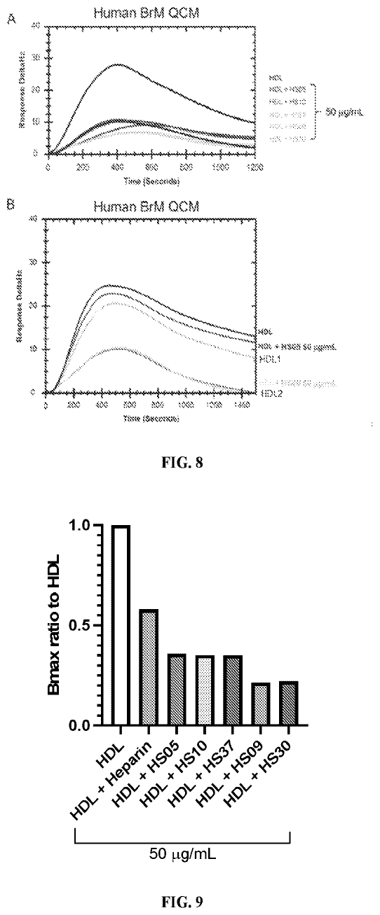

The ability to pharmacologically block the lipoprotein-HS interaction using heparan sulfate and heparin was demonstrated, as evidenced in FIGS. 4, 5, and 7. Full-length HS chains appear to have a more significant impact on this interaction (FIGS. 8 and 9). Initially, it was shown that heparan sulfate is capable of removing lipoproteins from post-mortem BrM in AMD patients (FIGS. 4 and 5). It was shown that heparan sulfate is also capable of inhibiting the binding of HDL particles to BrM via QCM (FIG. 6). Given the significant side effect profile associated with anticoagulant heparp, the role of defined non-anticoagulant full-length HS species was investigated (Table 1). Interestingly, full-length HS species with no anticoagulant activity were also capable of inhibiting HDL binding to human BrM (FIGS. 7 and 8). Furthermore, they inhibit HDL binding belier than pharmaceutical grade heparin (FIG. 9).

TABLE 1
Heparin and Heparanoids

| | Avg. | | | | | | |
|---|---|---|---|---|---|---|---|
| | Molecular | | | | | | |
| | Weight | | | 2-O- | 6-O- | 3-O- | |
| Heparinoid | (kDa) | GlcNAc | GlcNS | sulfation | sulfation | sulfation | Aglycone |
| | | Approximate Number of groups/100 disaccharides | | | | | |
| Heparin | ~14 | 15 | 85 | 78 | 90 | 1-3 | None |
| (therapeutic) | | | | | | | |
| TEGA HS05 | ~35 | 100 | 0 | 0 | 3 | 0 | None |
| TEGA HS09 | ~20 | 10 | 90 | 70 | 95 | 0 | None |
| TEGA HS10 | >40 | 5 | 95 | 2 | 4 | 0 | None |
| TEGA HS30 | ~25 | 15 | 85 | 65 | 90 | 1-3 | None |
| TEGA HS37 | ~20 | 5 | 95 | 0 | 60 | | None |
| | | groups/12-mer | | | | | |
| NAc 12-mer^{a} | 2414 Da | 6 | 0 | 0 | 0 | 0 | pNp |
| NS2S 12-mer^{b} | 2963 Da | 0 | 6 | 4 | 0 | 0 | pNp |
| NS2S6S3S12-mer^{b} | 3523 Da | 0 | 6 | 4 | 6 | 1 | pNp |

pNp, para-nitrophenol;
GlcNAc, N-acetyl glucosamine;
GlcNS, N-sulfo glucosamine
^{a}contains-6-GlcA, O IdoA;
^{b}contains 2-GlcA, 4 IdoA

In Table 1 above, the NAc 12-mer refers to a glycan having the following structure: GlcNS6S-GlcA-GlcNS3S6S-IdoA2S-GlcNS6S-IdoA2S-GlcNS6S-IdoA2S-GlcNS 6S-IdoA2S-GlcNS6S-GlcA-pNP (MW=3523): NS2S 12-mer refers to a glycan having the following structure: GlcNS-GlcA-GlcNS-GlcA-GlcNS-GlcA-GlcNS-GlcA-G 1cNS-GlcA-GlcNS-GlcA-pNP (MW=2643); and NS2S6S3S 12-mer refers to a glycan having the following structure: GlcNS-GlcA-GlcNS-IdoA2S-GlcNS-IdoA2S-GlcNS-IdoA 2S-GlcNS-IdoA2S-GlcNS-GlcA-pNP (MW=2963).

In vivo pharmacokinetics of heparin: Delivery of pharmaceuticals to the intraocular space can impede pharmaceutical development. The primary intraocular injections performed in office procedures clinically is intravitreal injections and surgically through subretinal injections. The pharmacokinetics of heparin via intraocular injection using BODIPY-labeled heparin was tested. The results show that intravitreal injection and not subretinal injection is capable of delivering heparin directly to the Bruch's membrane (FIG. 10). Specifically. FIG. 10 shows the presence of BODIPY label present in the subretinal space following subretinal injection (right), whereas the BODIPY signal is present on the BrM following intravitreal injection (middle).

### CONCLUSION

These innovative findings and methodologies represent a significant advancement in understanding AMD pathogenesis and provide a new promising avenue for developing targeted therapies aimed at modulating lipoprotein-HS interactions in the macular region. Importantly, the instant disclosure describes new therapeutic interventions aimed at providing new treatments for AMD and related diseases.

### REFERENCES

1. Stone. E. M., et al., A single EFEMP1 mutation associated with both Malattia Leventinese and Doyne honeycomb retinal dystrophy. Nat Genet, 1999. 22(2): p. 199-202.
2. Zayas-Santiago, A., et al., Mutant Fibulin-3 Causes Proteoglycan Accumulation and Impaired Diffusion Across Bruch's Membrane. Invest Ophthalmol Vis Sci, 2017. 58(7): p. 3046-3054.
3. Weber, B. H., et al., Mutations in the tissue inhibitor of metalloproteinases-3 (TIMP3) in patients with Sorsby's fundus dystrophy. Nat Genet. 1994. 8(4): p. 352-6.
4. Hayward, C., et al., Mutation in a short-chain collagen gene, CTRP5, results in extracellular deposit formation in late-onset retinal degeneration: a genetic model for age-related macular degeneration. Hum Mol Genet, 2003. 12(20): p. 2657-67.
5. Hess, K., et al., Mesopic and Scotopic Light Sensitivity and Its Microstructural Correlates in Pseudoxanthoma Elasticum. JAMA Ophthalmol, 2020. 138(12): p. 1272-1279.
6. Loewinger, A. S., et al., Choriocapillaris Flow Signal Impairment in Patients With Pseudoxanthoma Elasticum. Invest Ophthalmol Vis Sci, 2023. 64(2): p. 21.
7. Gliem, M., et al., Reticular pseudodrusen associated with a diseased bruch membrane in pseudoxanthoma elasticum. JAMA Ophthalmol, 2015. 133(5): p. 581-8.
8. Clark, S. J., et al., Bruch's Membrane Compartmentalizes Complement Regulation in the Eye with Implications for Therapeutic Design in Age-Related Macular Degeneration. Front Immunol, 2017. 8: p. 1778.
9. Hageman, G. S., et al., A common haplotype in the complement regulatory gene factor H (HF1/CFH) predisposes individuals to age-related macular degeneration. Proc Natl Acad Sci USA, 2005. 102(20): p. 7227-32.
10. Raychaudhuri, S., et al., A rare penetrant mutation in CFH confers high risk of age-related macular degeneration. Nat Genet, 2011. 43(12): p. 1232-6.
11. Klein, R. J., et al., Complement factor H polymorphism in age-related macular degeneration. Science, 2005. 308(5720): p. 385-9.
12. Edwards, A. O., et al., Complement factor H polymorphism and age-related macular degeneration. Science, 2005. 308(5720): p. 421-4.
13. Haines, J. L., et al., Complement factor H variant increases the risk of age-related macular degeneration. Science, 2005. 308(5720): p. 419-21.
14. Williams, B. L., et al., Chromosome 10q26-driven age-related macular degeneration is associated with reduced levels of HTRA1 in human retinal pigment epithelium. Proc Natl Acad Sci USA, 2021. 118(30).
15. Yu, Y., et al., Prospective assessment of genetic effects on progression to different stages of age-related macular degeneration using multistate Markov models. Invest Ophthalmol Vis Sci, 2012. 53(3): p. 1548-56.
16. Fritsche. L. G., et al., Seven new loci associated with age-related macular degeneration. Nat Genet, 2013. 45(4): p. 433-9, 439e1-2.
17. Ratnapriya, R., et al., Rare and common variants in extracellular matrix gene Fibrillin 2 (FBN2) are associated with macular degeneration. Hum Mol Genet, 2014. 23(21): p. 5827-37.
18. Chen, W., et al., Genetic variants near TIMP3 and high-density lipoprotein-associated loci influence susceptibility to age-related macular degeneration. Proc Natl Acad Sci USA, 2010. 107(16): p. 7401-6.
19. Ferris, F. L., et al., A simplified severity scale for age-related macular degeneration: AREDS Report No. 18. Arch Ophthalmol, 2005. 123(11): p. 1570-4.
20. Ferris, F. L., 3rd, et al., Clinical classification of age-related macular degeneration. Ophthalmology, 2013. 120(4): p. 844-51.
21. Au, A., et al., Relationship Between Drusen Height and OCT Biomarkers of Atrophy in Non-Neovascular AMD. Invest Ophthalmol Vis Sci. 2022. 63(11): p. 24.
22. Hartmann. K. I., et al., Scanning laser ophthalmoscope imaging stabilized microperimetry in dry age-related macular degeneration. Retina. 2011. 31(7): p. 1323-31.
23. Wu, Z., et al., Microperimetry of nascent geographic atrophy in age-related macular degeneration. Invest Ophthalmol Vis Sci, 2014. 56(1): p. 115-21.
24. Wu. Z., et al., Fundus autofluorescence characteristics of nascent geographic atrophy in age-related macular degeneration. Invest Ophthalmol Vis Sci, 2015. 56(3): p. 1546-52.
25. Wu, Z., et al., Prospective Longitudinal Evaluation of Nascent Geographic Atrophy in Age-Related Macular Degeneration. Ophthalmol Retina, 2020. 4(6): p. 568-575.
26. Zhang, Q., et al., Comparison of histologic findings in age-related macular degeneration with RPE flatmount images. Mol Vis, 2019. 25: p. 70-78.
27. Chen, L., et al., Biometrics, Impact, and Significance of Basal Linear Deposit and Subretinal Drusenoid Deposit in Age-Related Macular Degeneration. Invest Ophthalmol Vis Sci, 2021. 62(1): p. 33.
28. Weismann, D., et al., Complement factor H binds malondialdehyde epitopes and protects from oxidative stress. Nature, 2011. 478(7367): p. 76-81.
29. Ebrahimi, K. B., et al., Oxidized low-density-lipoprotein-induced injury in retinal pigment epithelium alters expression of the membrane complement regulatory factors CD46 and CD59 through exosomal and apoptotic bleb release. Adv Exp Med Biol, 2014. 801: p. 259-65.
30. Shaw, P. X., et al., Complement factor H genotypes impact risk of age-related macular degeneration by interaction with oxidized phospholipids. Proc Natl Acad Sci USA, 2012. 109(34): p. 13757-62.
31. Wang, L., et al., Abundant lipid andprotein components of drusen. PLoS One, 2010. 5(4): p. e10329.
32. Curcio, C. A., Soft Drusen in Age-Related Macular Degeneration: Biology and Targeting Via the Oil Spill Strategies. Invest Ophthalmol Vis Sci, 2018. 59(4): p. AMD160-AMD181.
33. Curcio, C. A., et al., The oil spill in ageing Bruch membrane. Br J Ophthalmol, 2011. 95(12): p. 1638-45.
34. Curcio, C. A. and C. L. Millican, Basal linear deposit and large drusen are specific for early age-related maculopathy. Arch Ophthalmol, 1999. 117(3): p. 329-39.
35. Huang, J. D., C. A. Curcio, and M. Johnson, Morphometric analysis of lipoprotein-like particle accumulation in aging human macular Bruch's membrane. Invest Ophthalmol Vis Sci, 2008. 49(6): p. 2721-7.
36. Li. C. M., et al., Distribution and composition of esterified and unesterified cholesterol in extra-macular drusen. Exp Eye Res, 2007. 85(2): p. 192-201.
37. Malek, G., et al., Apolipoprotein B in cholesterol-containing drusen and basal deposits of human eyes with age-related maculopathy. Am J Pathol, 2003. 162(2): p. 413-25.
38. Karwatowski, W. S., et al., Preparation of Bruch's membrane and analysis of the age-related changes in the structural collagens. Br J Ophthalmol. 1995. 79(10): p. 944-52.
39. Marshall, G. E., et al., Type IV collagen and laminin in Bruch's membrane and basal linear deposit in the human macula. Br J Ophthalmol, 1992. 76(10): p. 607-14.
40. Das, A., et al., Ultrastructural localization of extracellular matrix components in human retinal vessels and Bruch's membrane. Arch Ophthalmol, 1990. 108(3): p. 421-9.
41. Aisenbrey, S., et al., Retinal pigment epithelial cells synthesize laminins, including laminin 5, and adhere to them through alpha3- and alpha6-containing integrins. Invest Ophthalmol Vis Sci, 2006. 47(12): p. 5537-44.
42. Call, T. W. and J. G. Hollyfield, Sulfated proteoglycans in Bruch's membrane of the human eye: localization and characterization using cupromeronic blue. Exp Eye Res, 1990. 51(4): p. 451-62.
43. Keenan, T. D., et al., Age-dependent changes in heparan sulfate in human Bruch's membrane: implications for age-related macular degeneration. Invest Ophthalmol Vis Sci, 2014. 55(8): p. 5370-9.
44. Kelly, U., et al., Heparan sulfate, including that in Bruch's membrane, inhibits the complement alternative pathway: implications for age-related macular degeneration. J Immunol, 2010. 185(9): p. 5486-94.
45. Kelly, U. L., et al., High-density lipoproteins are a potential therapeutic target for age-related macular degeneration. J Biol Chem, 2020. 295(39): p. 13601-13616.
46. Skalen, K., et al., Subendothelial retention of atherogenic lipoproteins in early atherosclerosis. Nature, 2002. 417(6890): p. 750-4.
47. Tabas. I., K. J. Williams, and J. Boren. Subendothelial lipoprotein retention as the initiating process in atherosclerosis: update and therapeutic implications. Circulation, 2007. 116(16): p. 1832-44.
48. Chait, A. and T. N. Wight, Interaction of native and modified low-density lipoproteins with extracellular matrix. Curr Opin Lipidol, 2000. 11(5): p. 457-63.
49. Schwenke, D. C. and R. W. St Clair, Influx, efflux, and accumulation of LDL in normal arterial areas and atherosclerotic lesions of white Cameau pigeons with naturally occurring and cholesterol-aggravated aortic atherosclerosis. Arterioscler Thromb, 1993. 13(9): p. 1368-81.
50. Williams, K. J. and I. Tabas, The response-to-retention hypothesis of early atherogenesis. Arterioscler Thromb Vasc Biol, 1995. 15(5): p. 551-61.
51. Gonzales, J. C., et al., Apolipoproteins E and AV mediate lipoprotein clearance by hepatic proteoglycans. J Clin Invest, 2013. 123(6): p. 2742-51.
52. Khalil, M. F., W. D. Wagner, and I. J. Goldberg, Molecular interactions leading to lipoprotein retention and the initiation of atherosclerosis. Arterioscler Thromb Vasc Biol, 2004. 24(12): p. 2211-8.
53. Libeu, C. P., et al., New insights into the heparan sulfate proteoglycan-binding activity of apolipoprotein E. J Biol Chem, 2001. 276(42): p. 39138-44.
54. MacArthur, J. M., et al., Liver heparan sulfate proteoglycans mediate clearance of triglyceride-rich lipoproteins independently of LDL receptor family members. J Clin Invest, 2007. 117(1): p. 153-64.
55. Olsson, U., G. Ostergren-Lunden, and J. Moses, Glycosaminoglycan-lipoprotein interaction. Glycoconj J, 2001. 18(10): p. 789-97.
56. Hurt-Camejo, E., et al., Cellular consequences of the association of apoB lipoproteins with proteoglycans. Potential contribution to atherogenesis. Arterioscler Thromb Vasc Biol, 1997. 17(6): p. 1011-7.
57. Olsson, U., et al., Possible functional interactions of apolipoprotein B-100 segments that associate with cell proteoglycans and the ApoB/E receptor. Arterioscler Thromb Vasc Biol, 1997. 17(1): p. 149-55.
58. Stanford, K. I., et al., Heparan sulfate 2-O-sulfotransferase is required for triglyceride-rich lipoprotein clearance. J Biol Chem, 2010. 285(1): p. 286-94.
59. David. G., et al., Developmental changes in heparan sulfate expression: in situ detection with mAbs. J Cell Biol. 1992. 119(4): p. 961-75.
60. Olsen, T. W. and X. Feng, The Minnesota Grading System of eye bank eyes for age-related macular degeneration. Invest Ophthalmol Vis Sci, 2004. 45(12): p. 4484-90.
61. Olsen, T. W., et al., The Nine-Step Minnesota Grading System for Eyebank Eyes With Age Related Macular Degeneration: A Systematic Approach to Study Disease Stages. Invest Ophthalmol Vis Sci, 2017. 58(12): p. 5497-5506.
62. McHarg, S., et al., Enrichment of Bruch's Membrane from Human Donor Eyes. J Vis Exp, 2015(105).
63. Lawrence, R., et al., Evolutionary differences in glycosaminoglycan fine structure detected by quantitative glycan reductive isotope labeling. J Biol Chem, 2008. 283(48): p. 33674-84.

What is claimed is:

1. A method of preventing, delaying the progression of, or treating age-related macular degeneration (AMD) comprising administering to a subject in need thereof an effective amount of a composition effective to inhibit lipoprotein binding to an extracellular matrix of the eye.

2. The method of claim 1, wherein the composition is effective to prevent accumulation of lipoprotein on the extracellular matrix.

3. The method of claim 1, wherein the composition is effective to remove lipoproteins from the extracellular matrix of the subject.

4. The method of claim 1, wherein the extracellular matrix is Bruch's membrane.

5. The method of claim 1, wherein the composition inhibits the formation of drusen on Bruch's membrane.

6. The method of claim 1, wherein the composition comprises a competitive inhibitor of lipoprotein binding to glycosaminoglycans of the extracellular matrix.

7. The method of claim 1, wherein the composition comprises a soluble glycan.

8. The method of claim 7, wherein the soluble glycan is an ionic glycan.

9. The method of claim 1, wherein the composition comprises heparan sulfate or a molecular mimetic thereof.

10. The method of claim 9, wherein the molecular mimetic of heparan sulfate is selected from alginate polymers, agarose polymers, cellulose polymers, chitosan polymers, dextran polymers, heparan polymers, hyaluronate polymers, starch polymers, carboxylated derivatives thereof, and sulfated derivatives thereof.

11. The method of claim 9, wherein the heparan sulfate or the molecular mimetic thereof has a molecular weight of at least about 5 kDa.

12. The method of claim 9, wherein the heparan sulfate or the molecular mimetic thereof has a molecular weight of at least about 20 kDa.

13. The method of claim 9, wherein the heparan sulfate or molecular mimetic thereof is a non-anticoagulant.

14. The method of claim 1, wherein the composition comprises a heparan sulfate or heparin binding molecule selected from surfen, protamine sulfate, a calixarene, caltrop-like multicationic molecule, and a poly-cationic peptide.

15. The method of claim 1, wherein the composition comprises an antibody or antigen binding fragment which disrupts lipoprotein binding to the extracellular matrix.

16. The method of claim 1, wherein the composition inhibits lipoprotein binding to the extracellular matrix by altering a structure of glycosaminoglycans (GAGs) of the extracellular matrix.

17. The method of claim 16, wherein the composition comprises a heparanase or sulfatase enzyme.

18. The method of claim 16, wherein the composition comprises a gene editing system configured to alter one or more genes encoding sulfotransferase enzymes of the subject.

19. The method of claim 1, wherein the AMD is early or intermediate stage AMD.

20. The method of claim 1, wherein the AMD is advanced AMD.

21. The method of claim 1, wherein the AMD comprises geographic atrophy and/or choroidal neovascularization.

22. The method of claim 1, wherein the composition is administered intraocularly.

23. The method of claim 22, wherein the intraocular administration is intravitreal administration.

24. The method of claim 1, wherein the composition is a pharmaceutical composition formulated for intraocular administration.

25. A pharmaceutical composition comprising an amount of soluble glycan effective to inhibit lipoprotein binding to an extracellular matrix of the eye, and a pharmaceutically acceptable carrier.

26. The pharmaceutical composition of claim 25, wherein the soluble glycan comprises heparan sulfate or a molecular mimetic thereof.

27. The pharmaceutical composition of claim 25, wherein the soluble glycan is heparan sulfate.

28. The pharmaceutical composition of claim 25, wherein the soluble glycan has a molecular weight of at least 5 kDa.

29. The pharmaceutical composition of claim 25, wherein the pharmaceutical composition is formulated for intraocular administration.
